(19) United States
(12) Patent Application Publication
(10) Pub. No.: US 2006/0083766 A1
(43) Pub. Date: Apr. 20, 2006

(54) Anti-termite agent

(76) Inventors: Naonori Miyata, Chigasaki-shi (JP); Shinichi Ogino, Odawara-shi (JP); Makoto Ubukata, Sapporo-shi (JP); Yasumitsu Uraki, Sapporo-shi (JP)

Correspondence Address: WENDEROTH, LIND & PONACK, L.L.P., 2033 K STREET N. W., SUITE 800, WASHINGTON, DC, 20006-1021, US

(21) Appl. No.: 11/249,405

(22) Filed: Oct. 14, 2005

(30) Foreign Application Priority Data
Oct. 18, 2004 (JP) 2004-303118

(51) Int. Cl. A01N 25/08; C12N 9/08

(52) U.S. Cl. 424/410; 435/192

(57) ABSTRACT

Anti-termite agent which comprises lignin or modified compound thereof as the active ingredient.

This invention relates to an anti-termite agent or anti-termite composition for anti-termite treatment of rottable substrate surface such as of wood.

### BACKGROUND TECHNOLOGY

Normally sawn, dried and cut wood is subjected to, where necessary, an anti-termite treatment, to be protected from rotting by wood-rotting fungus or the like. Such an anti-termite treatment is occasionally given to wood at raw, unprocessed material stage, such as rectangular timber or board, or at the site of actual use of wood such as construction site.

Heretofore various chemicals have been used for anti-termite treatment, such as those derived from creosote oil, organic phosphor, azole, carbamate, pyrethroid, naphthalene, copper and the like. Some of those chemicals exhibit strong toxicity and are subject to such problems that they cannot be used for treating the parts with which men would contact, or, even when used for treating the parts which normally are not contacted by men, such as house foundations, the workers have to pay meticulous care not to contact the chemicals themselves or the wood which were given the anti-termite treatment. Furthermore, the effect of many of those chemicals has low durability and hence the treatment has to be repeated at an interval of several years, which gives rise to a cost problem. Still in addition, there is a possibility that the volatilized or eluted chemicals are detrimental to the environments or human bodies.

As anti-termite treatment methods of wood other than those using chemicals, for example, “Research on Nature-compatible Substance and Material Technology Directing Harmony with Nature”, Makoto OHKOSHI, Report on achievements by the studies (the first term) on establishing evaluation and design techniques for eco-materialization, 1997, pp. 317-326, reported a means for acetylating hydroxyl groups in wood components with acetic anhydride. According to that method, acetylated cellulose in the wood cannot be digested and absorbed by termites which took it in, and the termites are said to die of malnutrition. However, while acetylation of wood is found to show more durable effect of the treatment and higher safety, it requires the reaction with highly reactive acetic abhydride at high temperature, which makes it virtually impossible to carry out the anti-termite treatment at construction sites where safety cannot be secured.

As still other methods to prevent termite damage on wood, controlling under-the-floor temperature of houses or intercepting entering route of termite with a screen or the like have been practiced, but complete prevention cannot be expected from either of those means alone, but in most cases concurrent chemical treatment is required.

### DISCLOSURE OF THE INVENTION

The object of the present invention is to provide an anti-termite agent or anti-termite composition which exhibits very low toxicity, little volatilization or elution and allows ready anti-termite treatment at construction sites.

Chief components of wood are cellulose, hemicellulose and lignin, among which cellulose serves as the main nutritive source for growth of termite. Because termite likes places of relatively high humidity, taking houses for example, foundation parts under the floor where moisture is apt to gather and stay, in particular, wood around water distribution arrangement, most frequently suffer from termite damage. Such sites are normally at secret corners and early discovery of termite damage on the wood is extremely difficult except for experts. Progress in termite damage is liable to bring about very serious loss including collapse of the house building.

As a result of concentrative studies on means for preventing termite damage incurred on wood as above-described, we have now discovered that excellent ani-termite effect could be achieved by treating wood surfaces, by such means as application, impregnation or the like, using an anti-termite agent which contains lignin collected from plant body or a modified compounds thereof as the active ingredient, and come to complete the present invention.

Accordingly, therefore, the present invention provides an anti-termite agent characterized by comprising lignin or its modified compound as the active ingredient.

This invention also provides an anti-termite composition which comprises lignin or its modified compound and a diluent or carrier.

The invention furthermore provides a termite-controlling method characterized by applying to wood material, or impregnating the wood material with, above anti-termite agent or composition.

The anti-termite agent or anti-termite composition of the present invention has very low toxicity, hardly volatilizes or is eluted and hence allows ready practice of anti-termite treatment at construction sites. Lignin or modified compounds thereof are normally high molecular weight substances having number-average molecular weight no less than 1,000, unlike low molecular weight anti-termite agents used heretofore. Furthermore, they are basically water-insoluble and there is little fear of their loss by volatilization or elution after an anti-termite treatment using them. Thus durability over a very prolonged period of their anti-termite effect can be achieved. Still in addition, lignin or modified compounds thereof are of natural origin, very safe for human bodies, free of unpleasant odor like that of creosote oil although they have a unique smell, and are harmless to environments.

Furthermore, for wood which is expected to be used under severe conditions, lignin or modified compounds thereof can be made to form more potent treating coat, by concurrent use of a crosslinking agent having functional groups which are crosslinkable with functional groups present in lignin or modified compounds thereof, mainly phenolic and/or alcoholic hydroxyl groups.

Hereafter the present invention is explained in further details.

Lignin is a generic name for amorphous high molecular weight substances occurring in nature as cell wall component in vascular bundles of plants, in which phenylpropane-derived structural units are condensed in complex manner. They are conventionally classified into needle-leaved tree lignin, broad-leaved tree lignin and herbaceous plant lignin. Lignin can be separated or recovered from plant body by various methods, and lignin recovered by any of those methods can be used in the present invention. Depending on the separation and recovery method used, molecules of lignin may be cut, may form new linkages, or functional groups in the molecules may be chemically modified. Compounds resulting from such modifications are also useful in the present invention. In this invention, therefore, not only the modified compounds formed at the separation and recovery time of lignin from plant body but also further modified lignin compounds after the recovery by chemical means may be collectively referred to as “modified compounds.” Also in this specification, lignin and modified compounds thereof may be collectively referred to as “lignin compounds.”

In the present invention, lignin compounds separated and recovered from plant body by any method known per se can be used, such as, for example, insoluble lignins (e.g., sulfuric acid lignin, hydrochloric acid lignin and the like) which are obtained as insoluble residues after dissolving and removing from plant body all components other than lignin with conc. sulfuric acid, conc. hydrochloric acid or the like; soluble lignins obtained by chemically releasing lignin from plant body by delignification; and lignins obtained by extraction from plant body with an organic solvent (e.g., Björkman lignin). Furthermore, lignin compounds obtained by digestion of plant body with an aqueous solution of organic acid comprising phenol or its derivative or high temperature-boiling alcohol, or also a minor amount of inorganic acid as a catalyst are also useful. Of those methods, the digestion method using an aqueous organic acid solution can normally be carried out under ambient pressure and separation of lignin compounds from the digestion product is easy. Besides, thus obtained lignin compounds are organic acid-modified lignins which exhibit high solubility in organic solvent and hence are the optimum for use in the anti-termite agent or anti-termite composition of the present invention.

More specifically, the digestion with an aqueous organic acid solution can be carried out using a plant body and a solution containing an inorganic acid and a C₂-C₄ organic acid. The solution can be normally used in the form of an aqueous solution.

The inorganic acid acts as a catalyst for the digestion, examples of which include sulfuric acid, hydrochloric acid and nitric acid. In particular, sulfuric acid, hydrochloric acid or their mixtures at optional ratios are convenient. Its content can be suitably selected depending on such factors as the region or part of the plant body to be treated, the organic acid concentration in the treating solution and the reaction temperature. In general terms, a convenient range is 0.05-1 wt %, in particular, 0.1-0.5 wt %, to the whole solution. Where the inorganic acid content is less than 0.05 wt %, progress of the reaction is extremely slow. Conversely, where it exceeds 1 wt %, condensation of lignin progresses. In either of these cases yield of the organic acid-modified lignin markedly decreases, which is undesirable.

As C₂-C₄ organic acid, for example, monovalent carboxylic acids such as acetic acid, propionic acid, butyric acid and the like; polyvalent carboxylic acids such as succinic acid; and hydroxycarboxylic acid such as lactic acid can be named, which are useful either singly or in combination of two or more. Of those named above, acetic acid is particularly preferred, from the standpoints of boiling point, reactivity, smell and safety. Suitable organic acid content lies within a range of 60-99.95 wt %, in particular, 80-98 wt %, to the whole solution. Where the organic acid content is less than 60 wt %, reactivity between the organic acid and lignin is poor and lignin remains in the pulp, making it difficult to yield a sufficient amount of the organic acid-modified lignin. Besides, purity of cellulose of the pulp left after the digestion also decreases, which is undesirable in respect of total effective utilization of plant resources.

Plant bodies normally retain water content to a certain extent even in dry state. It is desirable, therefore, to measure water content of the plant material in advance and decide on the adequate organic acid concentration in the solution, taking the water content into consideration.

Digestion can be suitably carried out by the steps of drying the plant bodies where necessary, cut them into adequate sizes, adding thereto the solution in an amount of 5-20 times, in particular, 7-15 times, the dry weight of the plant bodies and heating at 90-140° C., preferably 100-120° C., for 2-8 hours, preferably 3-5 hours.

After the digestion, the inorganic acid is neutralized with an alkaline component such as sodium hydroxide or ammonia, and the system is filtered to recover pulp from the digestion product. So recovered pulp may be washed with an aqueous organic acid solution or the like where necessary. The filtrate and the washing are, for example, distilled under reduced pressure and concentrated to provide a concentrate which is then over-diluted with water, and the resulting precipitate is separated and dried to provide an organic acid-modified lignin. The drying is preferably carried out by reduced pressure-drying at temperatures not higher than 40° C. or by lyophilization, because high temperature drying is liable to promote condensation of the organic acid-modified lignin.

Any of plant bodies rich in lignin content can be used as the starting materials for separating and recovering lignin compounds, without limitation on their species. For example, needle-leaved trees represented by sugi (Japanese cedar), hinoki (Japanese cypress), matsu (pine tree), and the like; broad-leaved trees such as white birch, Japanese beech, eucalyptus and the like; and herbaceous plants such as kenaf, rice straw and the like can be used. These plant bodies may be in the state as lumbered from forests or mowed from farms, or cut up, crushed or ground. Scrap wood after use in buildings can also be used.

Lignin compounds used in the present invention in general often undergo breaking of chemical bonds during the separation and recovery stage, and can normally have number-average molecular weight within a range of about 800- about 10,000, in particular, about 1,000- about 4,000. Here the number-average molecular weight refers to the value obtained by converting a measured value by gel permeation chromatography using tetrahydrofuran as the solvent at a flow rate of 1.0 ml/min., based on number-average molecular weight of polystyrene. As the device for carrying out gel permeation chromatography, HLC8120GPC (tradename, Tosoh Corporation) can be used, and as the column therefor, TSKgel G-4000 H×L, TSKgel G-3000 H×L, TSKgel G-2500 H×L or TSKgel G-2000 H×L (tradename, Tosoh Corporation) can be used.

According to the invention the lignin compounds can be used as anti-termite agent as they are, while they are normally used in the form of anti-termite compositions or formulations which contain them in combination with diluent or carrier. More specifically, it is preferred to use them as liquid compositions as dissolved or dispersed in organic solvent or alkaline aqueous solution. In particular, most of above-described organic acid-modified lignins are soluble in not only polar solvent such as tetrahydrofuran, chloroform, dimethylsulfoxide, pyridine and the like, but also ketone-type organic solvent such as acetone, cyclohexanone and the like, while the solubility differs depending on the starting wood or the organic acid used, and they are also soluble in aqueous solutions of inorganic alkali such as sodium hydroxide or potassium hydroxide, or in those of certain kind of amines or aqueous ammonia. Hence, they can be applied onto the wood surfaces or used for impregnating the wood surfaces, in the form of such solutions.

The lignin compounds may also be mixed with solid diluent or carrier such as high molecular weight polyethylene glycol to form powdery compositions which can be dissolved or dispersed in organic solvent or alkaline aqueous solution as above-described, at the time of their application onto the wood surfaces.

It is generally desirable for the anti-termite agent or anti-termite composition according to the invention to contain the lignin compound at a concentration within a range of at least 35 wt %, in particular, 45-95 wt %, inter alia, 50-80 wt %.

Lignin compounds have phenolic hydroxyl groups and can self-crosslink by means of oxidative polymerization. Anti-termite agents or anti-termite compositions according to the present invention can be blended with catalyst and/or enzyme, which promote phenols' oxidative polymerization reaction, in one-package or two-package type, for more effectively enhance this self-crosslinking ability. Examples of the catalyst and enzyme include metal complex such as iron-salen complex and enzyme such as peroxidase, laccase and the like. Blending of such a catalyst and/or enzyme allows fixation of the anti-termite agent or anti-termite composition on the substrate surface within a short time, can increase the anti-termite effect and furthermore can improve durability of said effect.

Blend ratio of the catalyst and/or enzyme which promote the oxidative polymerization can lie within a range of 0.1-5 wt parts, preferably 0.5-3 wt parts, per 100 wt parts of solid component of lignin compounds.

The lignin compounds have not only phenolic hydroxyl groups but also alcoholic hydroxyl groups, and where necessary, a crosslinking agent having functional groups reactable with the alcoholic hydroxyl groups or phenolic hydroxyl groups can be blended in those anti-termite agents or anti-termite compositions according to the present invention. This enables to further enhance their anti-termite effect and prolong durability of the effect. Useful crosslinking agent includes, for example, isocyanate-containing compounds, epoxy-containing compounds, acid anhydrides and the like. Of these, isocyanate-containing compounds are preferred from the viewpoint of low temperature hardenability.

As examples of isocyanate-containing compounds, diisocyanate compounds such as hexamethylene diisocyanate, isophorone diisocyanate, toluidine diisocyanate, diphenylmethane diisocyanate and the like; and derivatives thereof such as biuret, isocyanurate, urethodione and the like can be named. Isocyanate groups in those isocyanate-containing compounds may be blocked with a blocking agent, where necessary.

As examples of epoxy-containing compounds, ethylene glycol diglycidyl ether, propylene glycol diglycidyl ether, polyethylene glycol diglycidyl ether, glycerol diglycidyl ether, epoxylated soybean oil, polyglycerol polyglycidyl ether, sorbitol polyglycidyl ether, pentaerythritol polyglycidyl ether, resorcine diglycidyl ether and the like can be named.

The blend ratio of such a crosslinking agent can normally lie within a range of 30-70 wt parts, preferably 40-60 wt parts, per 100 wt parts of solid component of the lignin compound.

Where these crosslinking agents are used, the anti-termite agents or anti-termite compositions of the present invention may be either one-package type or two-package type. In the latter case, it is desirable to keep the lignin compound component and crosslinking agent component separately and mix them at the time of use.

Compounds having functional groups such as isocyanate groups, epoxy groups, acid anhydride groups and the like can also be used as a modification agent for modifying lignin compounds, so modified lignin compounds being used as anti-termite component. The use rate of such a modification agent and reaction conditions should be adequately determined, paying minute care not to induce gelation. As the modification agents, besides those named in the above as examples of crosslinking agents, monoisocyanate compounds, monoepoxy compounds and the like can also be used.

In the occasions of using those crosslinking agents, further addition of a catalyst for promoting the crosslinking reaction assists fixation of anti-termite agents or anti-termite compositions of the invention onto substrate surfaces within a short time. Where an epoxy-containing compound is used as the crosslinking agent, for example, quaternary ammonium salt represented by tetrabutylammonium chloride can be used as the catalyst. Where an isocyanate-containing compound is used as the crosslinking agent, for example, tin compound such as dibutyltin dilaurate, acetylacetonate complex of divalent iron, a complex formed by coordinating dinitrobenzene with acetylacetonate complex of divalent iron, and the like can be used as the catalyst.

The blend ratio of such a catalyst can lie within a range of 0.05-2 wt parts, preferably 0.1-1 wt part, per 100 wt parts of the combined solid component of the lignin compound and crosslinking agent.

Where necessary, the anti-termite agent or anti-termite composition of the invention may further contain, besides the above-described components, for example, modifying resin such as thermoplastic resin, self-crosslinking resin, hydroxyl-containing resin other than lignin compounds and the like; additives such as pigment, dye, surfactant and the like; anti-termite chemicals other than lignin compounds; or the like as suitably blended. As examples of self-crosslinking resin, alkyd resin or fatty acid-modified acrylic resin which have oxidative polymerizability can be named.

Anti-termite treatment using an anti-termite agent or anti-termite composition of the present invention can be carried out by, for example, applying the agent or composition onto substrate surface or dipping the substrate in liquid anti-termite agent or anti-termite composition, according to the methods of operation known per se, whereby a protective film having excellent anti-termite effect and durability can be formed on the substrate surface. When the substrate is made of an easily liquid-permeable material like wood, better effect can be achieved by allowing the anti-termite agent or anti-termite composition to not only cover the surface but penetrate into inside of the material. To accomplish such a deep permeation of the agent or composition to inside of the substrate, such means as pressurization, pressure reduction or the like may be concurrently used.

As substrate materials to which anti-termite agent or anti-termite composition of the present invention is applicable, for example, wood, fibers, concrete, bricks, resin foam and the like can be named, wood being the most suitable.

The protective film as formed can be dried at ambient temperature or, where necessary, can be hardened by heating. In the latter case, it is desirable to suitably select the optimum heating temperature and heating time, taking into consideration the heat resistance of individual substrate material.

### EXAMPLES

Hereinafter the present invention is explained more specifically, referring to working examples, it being understood that the present invention is not limited to these examples only. In the examples, % and parts are by weight.

### Example 1

(1) Extraction of Organic Acid-Modified Lignin (Acetic Acid Lignin)

Nine (9) kg of white birch chips having a water content of 9.5% and 66.4 kg of 90% aqueous acetic acid solution containing 0.3% of sulfuric acid were charged in a heat reaction vessel equipped with a cooling tower and digested for 4 hours at reflux temperature. Thereafter the added sulfuric acid was neutralized with sodium hydroxide, and the system was cooled to room temperature. The liquid withdrawn from the reactor was concentrated under reduced pressure and over-diluted with distilled water. Lyophilizing the resulting precipitate, a brown powder (acetic acid lignin) was obtained. The acetic acid lignin yield to the dry weight of the white birch chips was 24.5%.

(2) Formulation of a Liquid Anti-Termite Agent

The acetic acid lignin as obtained in (1) above was dissolved in acetone to a solid concentration of 45% and the solution was used as a liquid anti-termite agent.

(3) Anti-Termite Test

Five Japanese cedar blocks of each 1 cm×1 cm at the cut end and 2 cm in length were used per test run. Onto the wood pieces the liquid anti-termite agent as obtained in (2) above was applied with a brush at an application rate of 40-50 g/m² as uniformly as possible, followed by a week's drying at room temperature. The wood pieces were then immersed in ten times their volume of 25° C. deionized water for 5 hours and dried at 40° C. for 19 hours. This immersion-drying cycle was repeated ten times to provide wooden test pieces.

On a part of an acrylic resin-made cylindrical vessel, 8 cm in diameter and 6 cm in height, dental hard gypsum was applied and hardened to a thickness of about 5 mm. On the gypsum forming a part of the vessel for rearing termite, about 1 mm-thick plastic net was placed and on which the wooden test pieces were placed with their cut ends coming at the top and bottom. Into the vessel 150 worker house termites and 15 soldier house termites were thrown, and the vessel was let stand in a 28±2° C. dark place for 21 days. The termites which died during the test term were quickly removed.

(4) Evaluation of Anti-Termite Performance

After the end of the termite damage test, the test pieces were withdrawn from the vessel, thoroughly removed of their surface deposits, air-dried for 24 hours, further dried at 60° C. for 48 hours and their dry weight was measured. The loss rate in their weight was calculated based on the same wooden test pieces' weight which had been measured in advance of the termite damage test, and anti-termite performance of the tested anti-termite agents was evaluated according to the following standard:

◯: weight loss rate was less than 3%,

Δ: weight loss rate was not less than 3% but less than 15%,

x: weight loss rate was at least 15%.

### Example 2

Example 1 was repeated except that a solution of the acetic acid lignin in 1N aqueous sodium hydroxide solution at a solid concentration of 25% was used as the liquid anti-termite agent which was applied onto the test pieces at an application rate of 30-40 g/m².

### Example 3

Example 1 was repeated except that a solution formed by dissolving the acetic acid lignin in acetone to a solid concentration of 30% and adding thereto 0.3% to the acetic acid lignin of N,N′-disalicylal-ethylenediamine iron (II) was used as the liquid anti-termite agent.

### Example 4

Example 1 was repeated except that a solution formed by dissolving 100 parts of acetic acid lignin, 60 parts of SUMIJULE N-3400 (tradename, Sumitomo Bayerurehtane Co.) and 0.3 part of dibutyltin (IV) dilaurate in acetone to a solid concentration of 40% was used as the liquid anti-termite agent.

### Example 5

Example 1 was repeated except that a solution formed by dissolving 100 parts of acetic acid lignin, 25 parts of polyethylene glycol diglycidyl ether and 0.5 part of tetrabutylammonium bromide in acetone to a solid concentration of 30% was used as the liquid anti-termite agent, and that the drying conditions of the wooden test pieces after application of the liquid anti-termite agent were changed to 3 hours at 110° C.

### Example 6

A flask was charged with 50 parts of xylene and 40 parts of 3-methoxybutyl acetate, which were heated up to 120° C. under stirring while passing gaseous nitrogen therethrough. The temperature was maintained at 120° C. during the following dropwise addition of a mixture of:

| | |
|---|---|
| styrene | 20 parts |
| i-butyl methacrylate | 15 parts |
| n-butyl acrylate | 45 parts |
| glycidyl methacrylate | 20 parts |
| 2,2′-azobisisobutyronitrile | 5 parts |

over 4 hours. Thereafter the reaction product was aged at 120° C. for 2 hours, the temperature was raised to 140° C., and an addition reaction with fatty acid was carried out by adding 33.3 parts of linseed oil fatty acid and 0.4 part of N,N′-dimethylaminoethanol as the catalyst. Tracing the acid value of the formed resin by KOH titration method, the reaction was terminated when the acid value dropped to not higher than 1.0. After termination of the reaction, the product was diluted with 45 parts of cyclohexanone to provide a nearly brown, transparent and viscous resin solution having a non-volatile component content of 50%.

A mixture of 100 parts of this resin solution, 20 parts of acetic acid lignin and 65 parts of acetone was used as the liquid anti-termite agent and otherwise all operations were conducted as in Example 1.

### Comparative Example 1

Example 1 was repeated except that the wooden test pieces were not treated with the liquid anti-termite agent.

The results of evaluation of anti-termite performance of the liquid anti-termite agents as obtained in Examples 1-6 and Comparative Example 1 were as shown in Table 1.

TABLE 1

| | Example | Example | Example | Example | Example | Example | Comparative |
|---|---|---|---|---|---|---|---|
| | 1 | 2 | 3 | 4 | 5 | 6 | Example 1 |
| Anti-termite | ∘ | ∘ | ∘ | ∘ | ∘ | ∘ | x |
| performance | | | | | | | |

1. An anti-termite agent characterized by comprising lignin or modified compound thereof as the active ingredient.

2. An anti-termite composition characterized by comprising lignin or a modified compound thereof, and a diluent or carrier.

3. An anti-termite agent or anti-termite composition as set forth in claim 1 or 2, in which lignin or a modified compound thereof is an organic acid-modified lignin obtained by digesting plant bodies with a solution containing 0.05-1% by weight of an inorganic acid and 60-99.95% by weight of a C₂-C₄ organic acid.

4. An anti-termite composition as set forth in claim 2, in which lignin or a modified compound thereof is dissolved or dispersed in an organic solvent.

5. An anti-termite composition as set forth in claim 2, in which lignin or a modified compound thereof is dissolved or dispersed in an alkaline aqueous solution.

6. An anti-termite agent or anti-termite composition as set forth in claim 1 or 2, which further comprises a catalyst and/or enzyme which promote oxidative polymerization reaction of phenols.

7. An anti-termite agent or anti-termite composition as set forth in claim 6, in which the catalyst is iron-salen complex and the enzyme is peroxidase or laccase.

8. An anti-termite agent or anti-termite composition as set forth in claim 6, which contains the catalyst and/or enzyme within a range of 0.1-5 parts by weight per 100 parts by weight of solid component of lignin or a modified compound thereof.

9. An anti-termite agent or anti-termite composition as set forth in claim 1 or 2, which further contains a crosslinking agent having functional groups reactable with alcoholic hydroxyl groups and/or phenolic hydroxyl groups in lignin or a modified compound thereof.

10. An anti-termite agent or anti-termite composition as set forth in claim 9, in which the crosslinking agent is an isocyanate-containing compound or an epoxy-containing compound.

11. An anti-termite agent or anti-termite composition as set forth in claim 9, which contains the crosslinking agent within a range of 30-70 parts by weight per 100 parts by weight of solid component of lignin or a modified compound thereof.

12. An anti-termite treatment method which comprises applying an anti-termite agent or anti-termite composition as set forth in claim 1 or 2 to a substrate, or impregnating the substrate with the agent or composition.

13. Anti-termite treated materials obtained by the method as set forth in claim 12.
